SEQUENCE LISTING (1 sequence)

SEQ ID NO: 1 - DNA, 1438 bp, Bacillus amyloliquefaciens
  Bacillus amyloliquefaciens
gggggctgctaagctgcaagtcgagcgggcagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttaggagccagccgccgaaggt

(19) United States
(12) Patent Application Publication
(10) Pub. No.: US 2022/0160794 A1
(43) Pub. Date: May 26, 2022

(54) USE OF BACILLUS AMYLOLIQUEFACIENS AND/OR BACILLUS SUBTILIS FOR THE ENHANCEMENT OF ACTIVE PROBIOTIC COMPOSITIONS OR FOR THE ACTIVATION OF INACTIVE PROBIOTIC COMPOSITIONS, COMPOSITIONS THUS OBTAINED, AND RELATED METHOD

(71) Applicant: ORMENDES SA, Jouxtens-Mezery (CH)

(72) Inventor: Stefano FIORUCCI, Perugia (IT)

(21) Appl. No.: 17/425,691

(22) PCT Filed: Apr. 29, 2020
(86) PCT No.: PCT/IB2020/054024

§ 371 Date: Jul. 23, 2021

(30) Foreign Application Priority Data
May 14, 2019 (IT) 102019000002567

(51) Int. Cl. A61K 35/745; A61K 35/747; A61K 45/06; A61K 35/742; C12N 1/20

(52) U.S. Cl. CPC A61K 35/745; A61K 35/747; C12R 2001/125; A61K 35/742; C12N 1/205; A61K 45/06

(57) ABSTRACT

This invention relates to the use of Bacillus amyloliquefaciens and/or Bacillus subtilis for the enhancement of active probiotic compositions or for the activation of inactive probiotic compositions for use in the prevention and/or treatment of inflammatory bowel diseases, such as Crohn's disease, ulcerative colitis, indeterminate colitis and microscopic colitis, the compositions thus obtained, and a method for the enhancement of active probiotic compositions or for the activation of inactive probiotic compositions for use in the prevention and/or treatment of inflammatory bowel diseases, such as Crohn's disease, ulcerative colitis, indeterminate colitis and microscopic colitis.

“Use of Bacillus amyloliquefaciens and/or Bacillus subtilis for the enhancement of active probiotic compositions or for the activation of inactive probiotic compositions, compositions thus obtained, and related method”

### FIELD OF THE INVENTION

This invention relates to the use of Bacillus amyloliquefaciens and/or Bacillus subtilis for the enhancement of active probiotic compositions or for the activation of inactive probiotic compositions for use in the prevention and/or treatment of inflammatory bowel diseases, such as Crohn's disease, ulcerative colitis, indeterminate colitis and microscopic colitis, the compositions thus obtained, and a method for the enhancement of active probiotic compositions or for the activation of inactive probiotic compositions for use in the prevention and/or treatment of inflammatory bowel diseases, such as Crohn's disease, ulcerative colitis, indeterminate colitis and microscopic colitis.

### BACKGROUND OF THE INVENTION

Chronic Inflammatory Bowel Diseases, designated with the English acronym IBD or with the Italian acronym MICI (Malattie Infiammatorie Croniche dell'Intestino) are pathological conditions with a high prevalence both in adult and paediatric subjects. To-date the available therapies adopted to treat such pathologies show only limited efficacy. Although IBD include different disease patterns, the two most common phenotypes of the disease are Crohn's disease (CD) and ulcerative colitis (UC). Both of these pathologies are chronic relapsing diseases supported by strong genetic predisposition and immunological pathogenesis, typically found in the intestine, although they can also affect other organs, taking into account that they can co-manifest in patients with autoimmune diseases, such as thyroiditis, sclerosing cholangitis and others (de Souza, H. S. and Fiocchi, C., Nat. Rev. Gastroenterol. Hepatol. 13 Jan. 2016(1):13-27).

Crohn's disease is a transmural intestinal inflammation that can affect any gastrointestinal tract, although the most common location is that affecting the terminal ileum alone or in association with colic location (ileocolic disease), which accounts for about 75% of all morbid forms.

Also ulcerative colitis can be defined as an intermittent inflammation, but which has not a transmural involvement. This condition is typical of the colon, and in particular of the terminal portion of the colon (proctitis). In 70% of cases, UC manifest only in the form of ulcerative proctitis (Danese, S. and Fiocchi. C., N. Engl. J. Med. 365:1713-1725, 2011).

Within the clinical symptomatology related to the presence of colitis, it is possible to identify a subgroup of patients with uncertain diagnosis, for whom the specific term of indeterminate colitis (IC) has been coined. This condition is characterized by an inflammation which, although limited to the colon, has histological peculiarities that can be attributed to both Crohn's disease and ulcerative colitis, but which do not allow an affiliation to either (Brown, C. J. et al., Dis. Colon Rectum 48(8):1542-9, 2005).

The term microscopic colitis finally identifies two disorders that are part of chronic inflammatory bowel diseases, characterized by chronic diarrhoea, namely collagenous colitis and lymphocytic colitis. Microscopic colitis mostly affects young women and is often associated with autoimmune diseases such as Sjögren's syndrome, celiac disease and some forms of arthritis. A significant increase in the incidence of microscopic colitis in individuals who chronically use certain drugs, such as non-steroidal anti-inflammatory drugs (NSAIDs) and proton pump inhibitors, has also been shown, which significantly alter the individual's microbiota.

There is now a general consensus that IBD derive from an alteration of the intestinal mucous barrier, which in genetically predisposed subjects leads to an overload of the local immune system with subsequent dysregulation of defense mechanisms involving both innate and adaptive immunity (Baumgart, D. C. and Sandborn, W. J., Lancet 380:1590-1605, 2011).

The current therapeutic treatment is typically based on the use of corticosteroids and immunosuppressants, such as azathioprine, and anti-inflammatory drugs, such as 5-aminosalicylate (Neurath, M. F., Nat. Rev. Gastroenterol. Hepatol. 14:269-278, 2017; Tiede, I. et al., J. Clin. Invest. 111:1133-1145, 2003; Lim, W. C. et al., Cochrane Database Syst. Rev. 7:CD008870, 2016). More recently, biological drugs with anti-TNF-α action, such as infliximab, adalimumab and golimumab, and anti-integrine, such as vedolizumab, have been introduced (Neurath, M. F., Nat. Rev. Gastroenterol. Hepatol. 14:269-278, 2017; Colombel, J. F. et al., N. Engl. J. Med. 362:1383-1395, 2010).

Although the drugs mentioned above are highly effective, to-date about half of patients tend to relapse during maintenance therapy and about 30-40% of patients, particularly those with CD, do not achieve complete remission, or do not permanently maintain remission, due to the lack of response to therapy or a reduced response over time.

Furthermore, not rarely, phenomena of intolerance to these drugs and/or the appearance of side effects occur, leading to the need to suspend therapy (Mosli, M. H. et al., Drugs 74:297-311, 2014).

In addition, therapeutic approaches targeting the regulation of leukocyte trafficking to the intestine through inhibition of integrins, chemokines and chemokine receptors have been developed, although today about 50% of patients relapse or do not respond to maintenance therapy (Mosli, M. H. et al., Drugs 74:297-311, 2014).

One of the study factors in the pathogenesis of IBD that is becoming increasingly important in recent years is represented by the intestinal microbiota. The intestinal microbiota constitutes the community of microbes comprising bacteria, fungi, archeobacteria, protozoa and viruses that colonize a specific environment, in this case the intestine, over a given period of time.

Alterations in the composition of the intestinal microbiota, called “dysbiosis”, are observed in about 75% of patients suffering from CD and may play a pathogenic role in the development of this pathology in genetically predisposed subjects (Bellaguarda, E. and Chang, E. B., Curr. Gastroenterol. Rep. 17:15, 2015).

In this context, the intestinal microbiota is considered an important therapeutic target in the treatment of IBD.

It is known that the composition/function of the intestinal microbiota can be modulated by the use of appropriate probiotics (Chibbar, R. and Dielman, L. A., J. Clin. Gastroenterol. 49:S50-S55, 2015).

Among the numerous probiotic preparations available on the market, the one that will be hereinafter referred to as “De Simone formulation” (U.S. Pat. No. 5,716,615), distributed over time under the brand VSL#3 and, later, under the brands VIVOMIXX and VISBIOME, has been extensively studied and is recommended for the treatment of chronic pouchitis (Gionchetti, P. et al, Gastroenterology 119:305-309, 2000; Gionchetti, P. et al., Gastroenterology 124(5):1202-9, 2003; Shen, J. et al., Bowel Dis. 20:21-35, 2014). It is a formulation consisting of a mixture of eight different bacterial strains. However, recent in vitro and in vivo studies have shown a variability in the efficacy in reducing inflammation by similar formulations produced from different raw materials obtained by different culture processes (Cinque, B. et al., J Cell Physiol. 232(12):3530-3539, 2017; Biagioli, M. et al., Front. Pharmacol. 8:505, 2017).

It is therefore felt the need to increase scientific research concerning the intestinal microbiota, and to carry out new studies and experiments in order to create new compositions based on probiotics, or to enhance existing ones to obtain compositions that are increasingly effective in the prevention and/or treatment of inflammatory bowel diseases.

The Applicant has unexpectedly found and experienced that the use of Bacillus amyloliquefaciens and/or Bacillus subtilis allows for the enhancement of already active probiotic compositions as well as the activation of inactive probiotic compositions, resulting in innovative and easy-to-use probiotic compositions that have proven to be particularly suitable for successful use in the prevention and/or treatment of inflammatory bowel diseases such as Crohn's disease, ulcerative colitis, indeterminate colitis and microscopic colitis.

The compositions obtained using Bacillus amyloliquefaciens and/or Bacillus subtilis have the following advantages:

1) use of bacterial species with lower production costs than traditional probiotics and which, owing to their stability, can be added both to probiotics to be stored at room temperature and to those to be stored in a refrigerated environment, wherein probiotics are formulated in the form of both liquid compositions and granules;
2) modification of the functionality of probiotic mixtures with enhancement or activation of therapeutic efficacy;
3) potent modulation of the intestinal immune system;
4) potent modulation of enzymatic activity.

### SUMMARY OF THE INVENTION

The Applicant has unexpectedly found that the use of Bacillus amyloliquefaciens and/or Bacillus subtilis in probiotic mixtures, preferably in total percentages between 2 and 50%, allows to obtain probiotic blends with high or enhanced therapeutic efficacy. The inclusion of Bacillus amyloliquefaciens and/or Bacillus subtilis in probiotic mixtures can not only increase the functional activity and thus the therapeutic activity of already active probiotic mixtures but, surprisingly, it can also make active probiotic mixtures inactive in reducing intestinal inflammation in the various experimental models investigated. More specifically, the present invention is based on evidence that the addition of Bacillus amyloliquefaciens and/or Bacillus subtilis to a probiotic mixture preferably in an overall percentage between 2 and 50%, allows to obtain an increase in anti-inflammatory activity, measured by determining the value of CDAI (Colitis Disease Activity Index), of at least 30% compared to an equal composition without Bacillus amyloliquefaciens and/or Bacillus subtilis, or to obtain active probiotic mixtures if the initial composition was inactive.

An important advantage of compositions containing Bacillus amyloliquefaciens and/or Bacillus subtilis is therefore that the presence of these bacteria in probiotic activates/increases their functionality by acting as a probiotic.

A further important advantage related to the use of the compositions of this aspect of the invention, which contain Bacillus amyloliquefaciens and/or Bacillus subtilis, is the increase of the regulatory type intestinal cells (Treg) present within the Lamina propria of the colon, known for their anti-inflammatory function.

In a first aspect, the invention relates to the use of Bacillus amyloliquefaciens and/or Bacillus subtilis for the enhancement of active probiotic compositions or for the activation of inactive probiotic compositions in the prevention and/or treatment of inflammatory bowel diseases, such as Crohn's disease, ulcerative colitis, indeterminate colitis and microscopic colitis.

According to a first embodiment of this aspect, the amount by weight of Bacillus amyloliquefaciens and/or Bacillus subtilis added is between 2 and 50%.

According to a second embodiment of this aspect, the active composition of probiotics to be enhanced is a mixture of the following 8 bacterial strains commercially available under the brands VIVOMIXX and VISBIOME:

Lactobacillus paracasei DSM 24733,

Lactobacillus plantarum DSM 24730,

Lactobacillus acidophilus DSM 24735,

Lactobacillus delbrueckii subspecies bulgaricus DSM 24734,

Bifidobacterium longum DSM 24736,

Bifidobacterium infantis DSM 24737,

Bifidobacterium breve DSM 24732, and

Streptococcus thermophilus DSM 24731.

According to a third embodiment of this aspect, the active composition of probiotics to be enhanced includes:

from 20 to 30% by weight of Streptococcus thermophilus,

from 20 to 30% by weight of Lactobacillus casei,

from 30 to 20% by weight of Bifidobacterium breve, and

from 30 to 20% by weight of Bifidobacterium animalis subsp. lactis.

According to a fourth embodiment of this aspect, the inactive composition of probiotics to be activated is a mixture of the following bacterial strains:

Streptococcus thermophilus BT01,

Bifidobacterium breve BB02,

Bifidobacterium longum (recently reclassified as Bifidobacterium animalis subspecies lactis) BL03,

Bifidobacterium infantis (recently reclassified as Bifidobacterium animalis subspecies lactis) BI04,

Lactobacillus acidophilus BA05,

Lactobacillus plantarum BP06,

Lactobacillus paracasei BP07, and

Lactobacillus delbrueckii subspecies bulgaricus (recently reclassified as Lactobacillus helveticus) BD08.

According to a fifth embodiment of this aspect, Bacillus amyloliquefaciens is the known strain characterized by SEQ ID NO: 1 coding for 16S RNA.

In a second aspect, the invention relates to an enhanced active probiotic composition or an activated inactive probiotic composition comprising a total of from 2 to 50% by weight of Bacillus amyloliquefaciens and/or Bacillus subtilis added for use in the prevention and/or treatment of inflammatory bowel diseases, such as Crohn's disease, ulcerative colitis, indeterminate colitis and microscopic colitis.

According to a first embodiment of this aspect, the enhanced composition of probiotics includes from 98 to 50% by weight of a mixture of the following 8 bacterial strains:

Lactobacillus paracasei DSM 24733,

Lactobacillus plantarum DSM 24730,

Lactobacillus acidophilus DSM 24735,

Lactobacillus delbrueckii subspecies bulgaricus DSM 24734,

Bifidobacterium longum DSM 24736,

Bifidobacterium infantis DSM 24737,

Bifidobacterium breve DSM 24732,

Streptococcus thermophilus DSM 24731, and

from 2 to 50% by weight of Bacillus amyloliquefaciens and/or Bacillus subtilis, for use in the prevention and/or treatment of inflammatory bowel diseases such as Crohn's disease, ulcerative colitis, indeterminate colitis and microscopic colitis.

According to a second embodiment of this aspect, the activated composition of probiotics includes

from 20 to 30% by weight of Streptococcus thermophilus,

from 10 to 30% by weight of Lactobacillus casei,

from 10 to 15% by weight of Bifidobacterium breve,

from 10 to 15% by weight of Bifidobacterium animalis subsp. lactis, and

from 50 to 10% by weight of Bacillus amyloliquefaciens and/or Bacillus subtilis,

for use in the prevention and/or treatment of inflammatory bowel diseases such as Crohn's disease, ulcerative colitis, indeterminate colitis and microscopic colitis.

According to a third embodiment of this aspect, the enhanced composition of probiotics

from 98 to 50% by weight of a mixture of the following 8 bacterial strains:

Streptococcus thermophilus BT01,

Bifidobacterium breve BB02,

Bifidobacterium longum (recently reclassified as Bifidobacterium animalis subspecies lactis) BL03,

Bifidobacterium infantis (recently reclassified as Bifidobacterium animalis subspecies lactis) BI04,

Lactobacillus acidophilus BA05,

Lactobacillus plantarum BP06,

Lactobacillus paracasei BP07,

Lactobacillus delbrueckii subspecies bulgaricus (recently reclassified as Lactobacillus helveticus) BD08, and

from 2 to 50% by weight of Bacillus amyloliquefaciens and/or Bacillus subtilis,

for use in the prevention and/or treatment of inflammatory bowel diseases such as Crohn's disease, ulcerative colitis, indeterminate colitis and microscopic colitis.

According to a fourth embodiment of this aspect, Bacillus amyloliquefaciens is the known strain characterized by SEQ ID NO: 1 coding for 16S RNA.

In a third aspect, the invention relates to a new composition of probiotics that includes:

from 20 to 30% by weight of Streptococcus thermophilus,

from 20 to 30% by weight of Lactobacillus casei,

from 30 to 20% by weight of Bifidobacterium breve, and

from 30 to 20% by weight of Bifidobacterium animalis subsp. lactis,

for use in the prevention and/or treatment of inflammatory bowel diseases such as Crohn's disease, ulcerative colitis, indeterminate colitis and microscopic colitis.

In a fourth aspect, the invention relates to a method for the enhancement of active probiotic compositions or for the activation of inactive probiotic compositions in the prevention and/or treatment of inflammatory bowel diseases, such as Crohn's disease, ulcerative colitis, indeterminate colitis and microscopic colitis, which includes the addition of Bacillus amyloliquefaciens and/or Bacillus subtilis to such probiotic compositions.

According to a first embodiment of this aspect, the quantity by weight of Bacillus amyloliquefaciens and/or Bacillus subtilis added is between 2 and 50%.

According to a second embodiment of this aspect, the active composition of probiotics to be enhanced is a mixture of the following 8 bacterial strains:

Lactobacillus paracasei DSM 24733,

Lactobacillus plantarum DSM 24730,

Lactobacillus acidophilus DSM 24735,

Lactobacillus delbrueckii subspecies bulgaricus DSM 24734,

Bifidobacterium longum DSM 24736,

Bifidobacterium infantis DSM 24737,

Bifidobacterium breve DSM 24732, and

Streptococcus thermophilus DSM 24731.

According to a third embodiment of this aspect, the active composition of probiotics to be enhanced includes:

from 20 to 30% by weight of Streptococcus thermophilus,

from 20 to 30% by weight of Lactobacillus casei,

from 30 to 20% by weight of Bifidobacterium breve, and

from 30 to 20% by weight of Bifidobacterium animalis subsp. lactis.

According to a fourth embodiment of this aspect, the inactive composition of probiotics to be activated is a mixture of the following 7 bacterial strains:

Streptococcus thermophilus BT01,

Bifidobacterium breve BB02,

Bifidobacterium longum (recently reclassified as Bifidobacterium animalis subspecies lactis) BL03,

Bifidobacterium infantis (recently reclassified as Bifidobacterium animalis subspecies lactis) BI04,

Lactobacillus acidophilus BA05,

Lactobacillus plantarum BP06,

Lactobacillus paracasei BP07, e

Lactobacillus delbrueckii subspecies bulgaricus (recently reclassified as Lactobacillus helveticus) BD08.

According to a fifth embodiment of this aspect, Bacillus amyloliquefaciens is the known strain characterized by SEQ ID NO: 1 coding for 16S RNA.

### BRIEF DESCRIPTION OF THE DRAWINGS

FIGS. 1A-1F show the enhancement of the protective effect associated with the administration of the species Bacillus amyloliquefaciens in addition to an already active bacterial composition with respect to this effect. In particular, FIG. 1A shows the trend of body weight, FIG. 1B shows the daily measurement of the Colitis Disease Activity Index (CDAI) which considers body weight loss, stool consistency and the presence of blood in stool, FIG. 1C shows the length of the colon, FIG. 1D shows the ratio of colon weight to colon length, and FIGS. 1E and 1F show the percentage of immune cells present in the Lamina propria of the colon (FIG. 1E: CD3+IL-10+ T lymphocytes within CD3+ T lymphocytes; FIG. 1F: M2 CD11b+Gr1-IL-10+ macrophages within CD11+Gr1-macrophages). The results are expressed as mean±SEM of 7-10 mice per experimental group. *p<0.05.

FIGS. 2A-2E show the enhancement of the protective effect associated with the administration of the bacterial species Bacillus amyloliquefaciens in addition to a further bacterial composition that is already active with respect to this effect. In particular, FIG. 2A shows the trend of body weight, FIG. 2B shows the daily measurement of CDAI that considers body weight loss, stool consistency and the presence of blood in stool, FIG. 2C shows the ratio of colon weight to colon length, and FIGS. 2D and 2E show the frequency of Treg CD4+FoxP3+ immune cells present in the Lamina propria of the colon (FIG. 2D: within CD4+ T lymphocytes; FIG. 2E: within all cells in the Lamina propria of the colon). The results are expressed as mean±SEM of 6-8 mice per experimental group. *p<0.05.

FIGS. 3A-3B show the protective effect associated with the administration of the Bacillus amyloliquefaciens species compared to a bacterial composition not presenting this effect. In particular, FIG. 3A shows the trend of body weight and FIG. 3B shows the daily measurement of CDAI which takes into account body weight loss, stool consistency and the presence of blood in stool. The results are expressed as mean±SEM of 6-8 mice per experimental group. *p<0.05.

FIGS. 4A-4B show the effect of Bacillus amyloliquefaciens alone as well as in combination with some of the compositions of the invention. In particular, FIG. 4A shows the trend of body weight and FIG. 4B shows the daily measurement of CDAI that considers body weight loss, stool consistency and the presence of blood in the stool. The results are expressed as mean±SEM of 6-8 mice per experimental group. *p<0.05.

FIGS. 5A-5B show the substantial equivalence of the effects of Bacillus amyloliquefaciens and Bacillus subtilis in the tests performed. In particular, FIG. 5A shows the trend of body weight and FIG. 5B shows the daily measurement of CDAI that considers body weight loss, stool consistency and the presence of blood in the stool. The results are expressed as mean±SEM of 6-8 mice per experimental group. *p<0.05.

### DETAILED DESCRIPTION OF THE INVENTION

### Experimental Part

Materials and Methods

The strain of Bacillus amyloliquefaciens used for the experiments is the known strain characterized by SEQ ID NO: 1 coding for 16S RNA.

The murine model of colitis induced by 2,4,6-trinitrobenzenesulfonic acid (TNBS) was used to test the anti-inflammatory activity of the compositions under examination. In this model, inflammation of the colon is induced by intrarectal administration of TNBS in a 50% ethanol solution. Ethanol is essential to provide access to intestinal epithelial cells, compromise barrier function and allow TNBS to penetrate the wall of the intestine.

Since this model is associated with the increased presence of highly activated T cells, which have a key pathogenic role in TNBS-induced colitis, the model itself is suitable to investigate the role of T cells in intestinal inflammation (Elson, C. O. et al., J. Immunol. 157:2174-2185, 1996; Dohi, T. et al., J. Exp. Med. 189:1169-1180, 1999). Nevertheless, immune mechanisms are also involved in the development of TNBS colitis (Fiorucci, S. et al., Immunity 17:769-780, 2002).

Histologically, following the induction of the disease, transmural inflammation with infiltrates of macrophages, neutrophils and lymphocytes, as well as colon hypertrophy (Kiesler, P. et al., Cell. Mol. Gastroenterol. Hepatol. 1:154-170, 2015) is obtained. Since some immunological and histopathological features are similar to those associated with Crohn's disease, TNBS-induced colitis has been widely used to study a variety of aspects potentially relevant to this disease (Kiesler, P. et al., Cell. Mol. Gastroenterol. Hepatol. 1:154-170, 2015; Rieder, F. et al., Gut 62:1072-1084, 2013).

C57BL6 male mice of 8 weeks of age purchased from The Jackson Laboratory were used. The animals were kept at the preclinical research center of the University of Perugia.

The mice were kept in controlled temperature of 22° C. with a light/dark period of 12/12 hours and were acclimatized under these conditions for 7 days before their use in experimental activities. The study was conducted in accordance with Italian law and the experimental protocol was approved by the Ethics Committee of the University of Perugia and the Ministry of Health (permit No. 1126/20-PR).

For the induction of colitis, mice were kept fasting for 12 h (Day −1). The next day (Day 0), the mice were anaesthetized, and a catheter was inserted into the colon up to 4 cm from the anus. To induce colitis, 1 mg of TNBS dissolved in 50% ethanol was administered by catheter with a 1 ml syringe (injection volume: 100 μl).

Control animals (untreated, NT) received only 50% ethanol solution. The mice were then monitored daily to assess the course of the disease by measuring body weight and the CDAI (Colitis Disease Activity Index) which includes the percentage of weight lost, stool consistency and the presence of blood in stool (each parameter has a value ranging from 0 to 4).

At the end of the experiment the animals were sacrificed, and the colon was recovered for the analysis of macroscopic characteristics and for the extraction of Lamina propria cells by means of the Lamina propria Dissociation Kit Mouse (Miltenyi Biotec) for analysis by cytofluorimetry.

The probiotic compositions were administered daily (from Day 0 to Day 4) by oral probe at a concentration of 50×10⁹ cfu of probiotics/kg body weight dissolved in saline solution.

### Example 1

In this example a composition active in the prevention and/or treatment of inflammatory diseases which includes the following 8 bacterial strains has been tested:

Lactobacillus paracasei DSM 24733,

Lactobacillus plantarum DSM 24730,

Lactobacillus acidophilus DSM 24735,

Lactobacillus delbrueckii subspecies bulgaricus DSM 24734,

Bifidobacterium longum DSM 24736,

Bifidobacterium infantis DSM 24737,

Bifidobacterium breve DSM 24732, and

Streptococcus thermophilus DSM 24731, commercially available under the VIVOMIXX brand, alone and with the addition of Bacillus amyloliquefaciens.

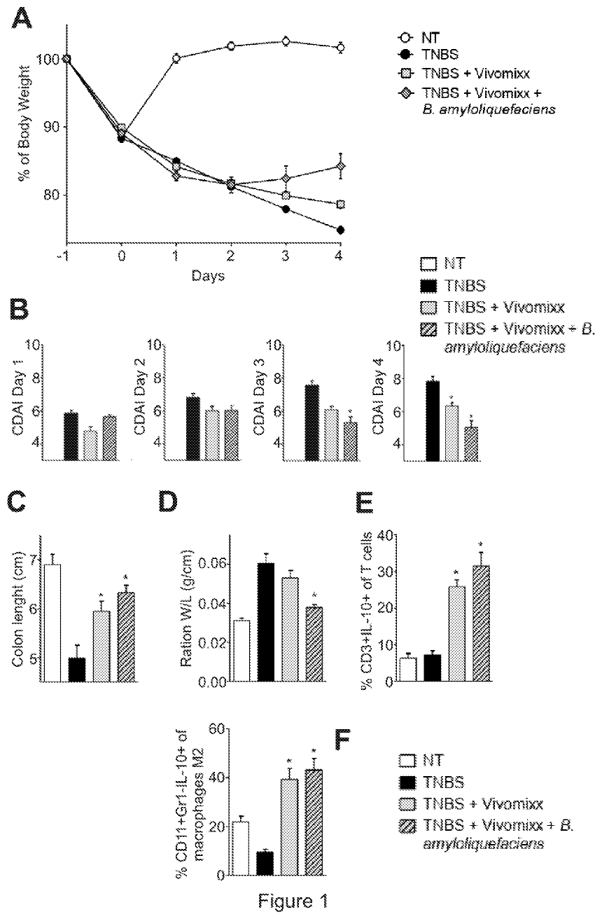

FIG. 1 shows the results of the tests carried out according to the methods described above. In the figure, NT=untreated and TNBS=treated with 2,4,6-trinitrobenzenesulfonic acid only.

The results of the experimental data clearly show that, in the TNBS-induced colitis model, the addition of Bacillus amyloliquefaciens improves the efficacy of VIVOMIXX in preventing the reduction of body weight loss, as well as in inducing improvements in inflammation measured as CDAI and in attenuating TNBS-induced colon morphological changes, such as the ratio of colon weight to colon length. The increase in regulatory type intestinal cells (Treg) within the Lamina propria of the colon is also evident.

### Example 2

In this example a new composition has been tested that has been found to be active in the prevention and/or treatment of inflammatory diseases.

The new composition included:

30% by weight of Streptococcus thermophilus,

30% by weight of Lactobacillus casei,

20% by weight of Bifidobacterium breve, and

20% by weight of Bifidobacterium animalis subsp. lactis.

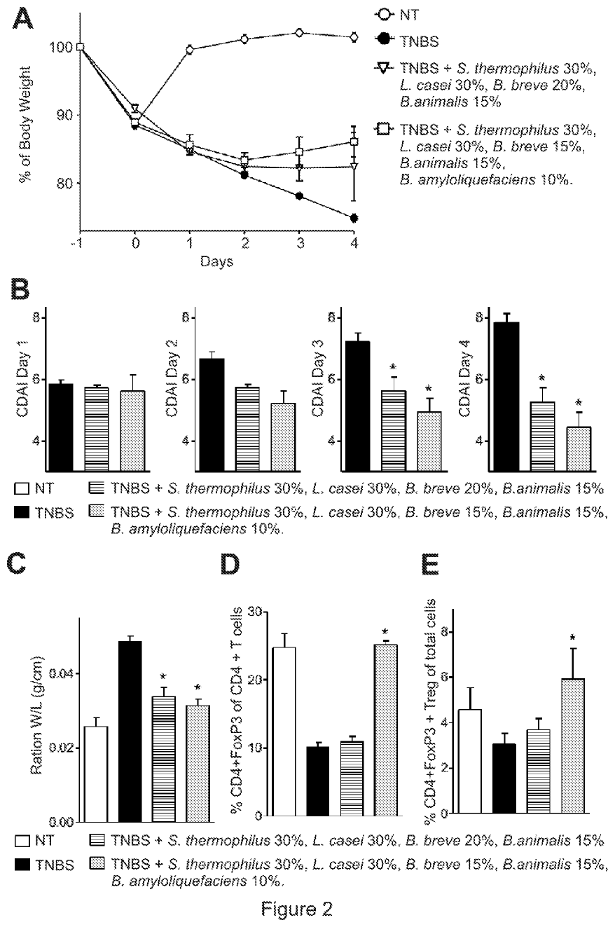

FIGS. 2A-2E show the results of the tests carried out according to the methods described above. In the figures, NT=untreated and TNBS=treated with 2,4,6-trinitrobenzenesulfonic acid only.

The results of the experimental data with the above composition clearly show that, in the TNBS-induced colitis model, this composition exhibits good efficacy in preventing the reduction of body weight loss as well as in inducing improvements in inflammation measured as CDAI and in attenuating TNBS-induced colon morphological changes, such as the ratio of colon weight to colon length. The increase in regulatory type intestinal cells (Treg) within the Lamina propria of the colon is also evident.

To verify the effects of adding Bacillus amyloliquefaciens to this composition, the composition has been modified as follows:

30% by weight of Streptococcus thermophilus,

30% by weight of Lactobacillus casei,

15% by weight of Bifidobacterium breve,

15% by weight of Bifidobacterium animalis subsp. lactis, and

10% by weight of Bacillus amyloliquefaciens.

The results of the experimental data summarised in FIGS. 2A-2E clearly show that, in the TNBS-induced colitis model, the addition of Bacillus amyloliquefaciens improves the effectiveness of the new composition in preventing the reduction of body weight loss, as well as in inducing improvements in inflammation measured as CDAI and in attenuating TNBS-induced colon morphological changes, such as the ratio of colon weight to colon length. The increase in regulatory type gut cells (Treg) within the Lamina propria of the colon is also evident.

### Example 3

In this example a commercial composition including the following bacterial strains has been tested:

Streptococcus thermophilus BT01,

Bifidobacterium breve BB02,

Bifidobacterium longum (recently reclassified as Bifidobacterium animalis subspecies lactis) BL03,

Bifidobacterium infantis (recently reclassified as Bifidobacterium animalis subspecies lactis) BI04,

Lactobacillus acidophilus BA05,

Lactobacillus plantarum BP06,

Lactobacillus paracasei BP07, e

Lactobacillus delbrueckii subspecies bulgaricus (recently reclassified as Lactobacillus helveticus) BD08.

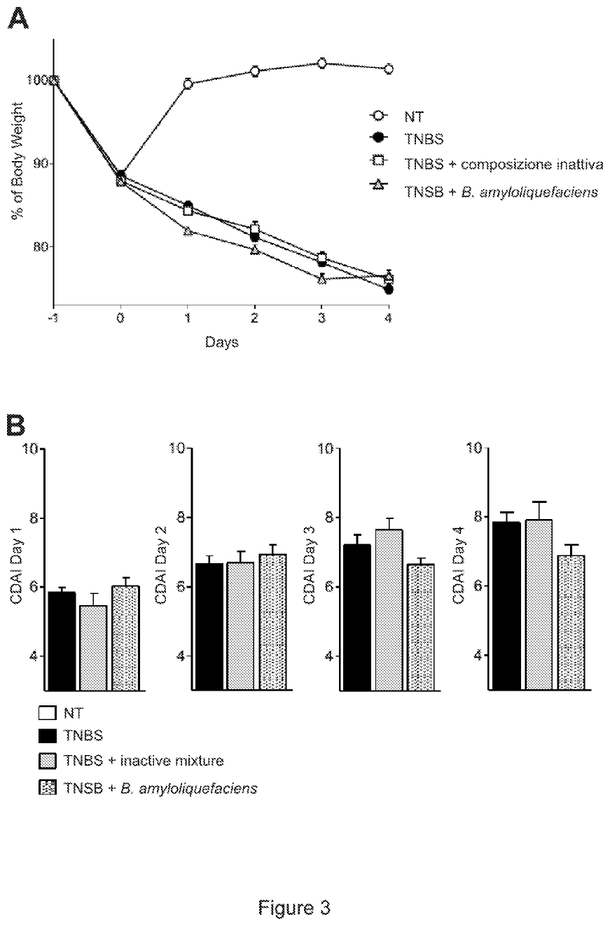

As can be seen from the results shown in FIG. 3, this composition was inactive in the tests performed, most likely due to differences in strain cultivation procedures.

Nevertheless, the addition of Bacillus amyloliquefaciens has made the composition active in preventing the reduction of body weight loss as well as in inducing an improvement in inflammation measured as CDAI.

### CONCLUSIONS

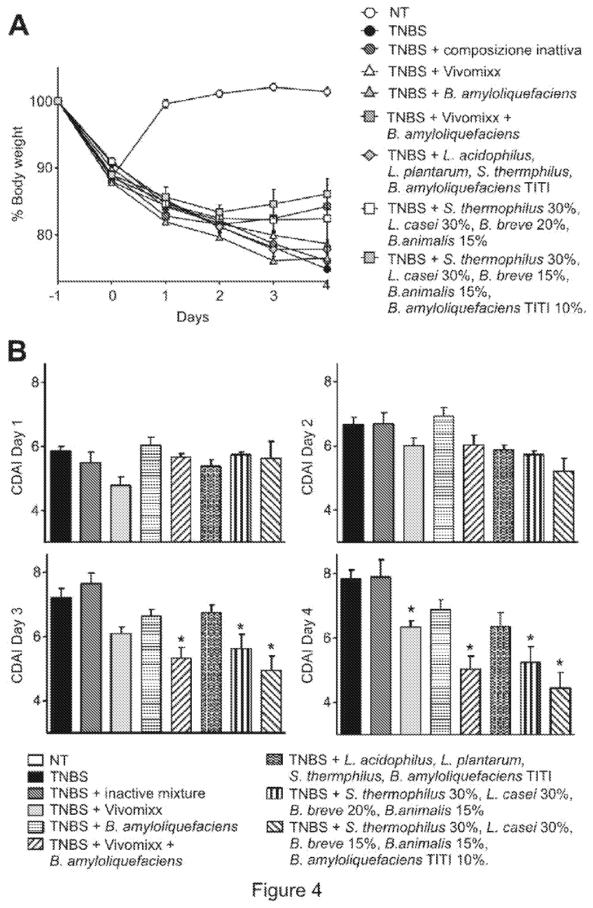

From FIGS. 4A-4B it is evident the enhancement of the protective effect associated with the administration of the bacterial species Bacillus amyloliquefaciens in addition to bacterial compositions already active with respect to this effect, or the establishment of the protective activity associated with the administration of the same species in addition to bacterial compositions originally not presenting this characteristic.

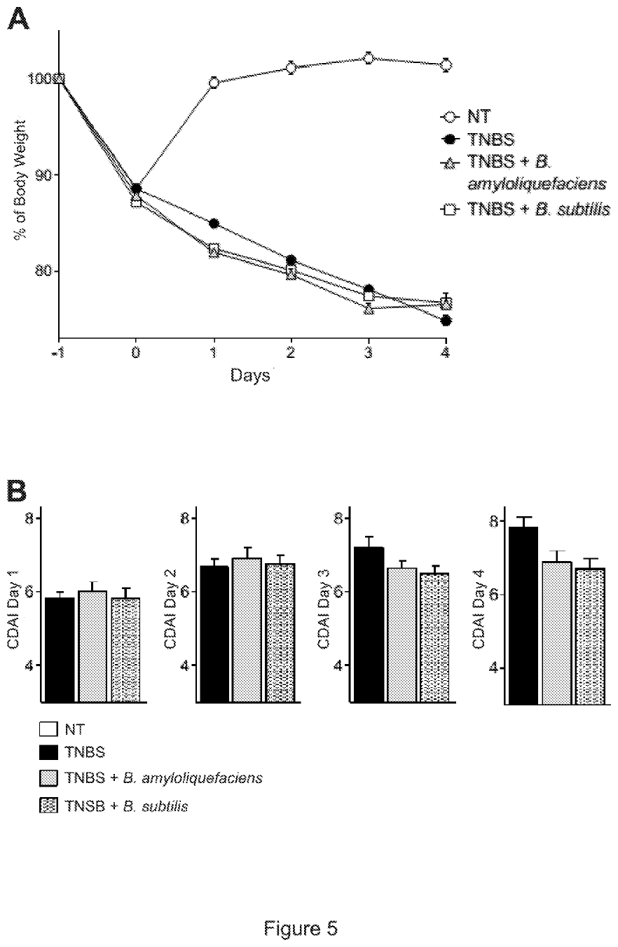

Furthermore, FIGS. 5A-5B show a substantial equivalence of the effects associated with the administration of the bacterial species Bacillus amyloliquefaciens and Bacillus subtilis. This equivalence would be explained by the high degree of genetic similarity between the two groups of bacteria.

In fact, Bacillus amyloliquefaciens and Bacillus subtilis constitute two sister species (species originated by differentiation from a shared ancestor) belonging to the larger group of bacterial species characterized by a high genetic correlation, called Bacillus subtilis group (Fritze, D., “Taxonomy of the genus Bacillus and related genera: the aerobic endospore-forming bacteria”, Phytopathology 94:1245-1248, 2004). The high genetic similarity between the species B. amyloliquefaciens and B. subtilis is clearly demonstrated by the difficulty and/or impossibility to efficiently distinguish these two bacterial groups by comparing the sequences encoding for 16S rRNA (Wang et al., “Bacillus velezensis is a later heterotypic synonym of Bacillus amyloliquefaciens”, Int. J. Syst. Evol. Microbiol. 58:671-675, 2008), which is the marker of choice for taxonomic identification of bacteria (Woese, C. R., “Bacterial evolution”, Microbiol. Rev. 51:221-271, 1987). In recent years, the development of new generation sequencing techniques made it possible to access a large amount of data concerning the sequence of the entire genome of many bacterial species and to develop investigation methods capable of assessing more reliably and accurately the genetic similarity between microbial groups. In this context, genome typing based on multiple loci extended to the entire preserved genome (core-MLST) represents a potent tool to determine the correlation between different taxonomic groups by means of the simultaneous comparison of hundreds or thousands of genetic markers shared between them. The application of this technique to specific bacterial strains belonging to the B. amyloliquefaciens and B. subtilis species further highlights the great genetic similarity between these taxonomic groups. The reconstruction of the phylogenetic relationships between genomes belonging to the two species, based on the alignment of 1664 gene clusters determined by using a sequence similarity threshold of ≥80% (see dendrogram below), shows that B. amyloliquefaciens and B. subtilis strains are phylogenetically closer to each other than to strains included in their own taxonomic group (groups A, B, C, D).

The high genetic correlation together with the large number of genes shared between the two bacterial species makes it possible to assume that they may also be equivalent from a functional point of view.

Materials and Methods

Genome typing based on multiple loci extended to the entire core-genome was performed on genomes related to the B. amyloliquefaciens and B. subtilis species available in the public database (ftp://ftp.ncbi.nlm.nih.gov/genbank). Five complete genomic sequences were selected for each bacterial species, including those related to the species type strains B. amyloliquefaciens DSM7 and B. subtilis ATCC13952. In order to avoid the introduction of errors due to different genome annotation, all sequences were annotated de novo by the Prokka v1.12 pipeline (Seeman T, “Prokka: rapid prokaryotic genome annotation”, Bioinformatics 15 Jul. 2014, 30(14):2068-9) forcing compliance with Genbank/ENA/DDJB standards. The genomes were imported within the BPGA bioinformatics program (Chaudhari et al., “BPGA-an ultra-fast pan-genome analysis pipeline” Sci. Rep. 6:24373, 2016) and homologous gene groups were determined using a threshold of similarity ≥80% between the protein sequences coded by the gene sequences. The homologous protein groups were aligned separately using the MUSCLE v 3.8.31 (Edgar, R. C., “MUSCLE: multiple sequence alignment with high accuracy and high throughput”, Nucleic Acids Res. 32:1792-7, 2004) and subsequently joined to form concatenameres. The phylogenetic reconstructions were performed using the Approximately Maximum-Likelihood method using the Fasttree 2 program (Price et al., “FastTree 2-Approximately maximum-likelihood trees for large alignments”, PLoS One 5:e9490, 2010).

FIGS. 5A-5B also show the substantial equivalence of the effects of Bacillus amyloliquefaciens and Bacillus subtilis in the tests performed.

1: A method for treating or ameliorating an inflammatory bowel disease comprising administering to an individual in need thereof a probiotic mixture in an amount sufficient to treat or ameliorate the inflammatory bowel disease in the individual in need thereof,
wherein the probiotic mixture comprises a Bacillus amyloliquefaciens and/or a Bacillus subtilis in an amount sufficient to enhance an active probiotic composition in the probiotic mixture or activate an inactive probiotic composition in the probiotic mixture,
thereby treating or ameliorating the inflammatory bowel disease.

2: The method of claim 1, wherein the amount by weight of Bacillus amyloliquefaciens and/or Bacillus subtilis in the probiotic mixture is between about 2 and about 50%.

3: The method of claim 1, wherein the active probiotic composition comprises a mixture of bacterial strains comprising:
Lactobacillus paracasei DSM 24733,
Lactobacillus plantarum DSM 24730,
Lactobacillus acidophilus DSM 24735,
Lactobacillus delbrueckii subspecies bulgaricus DSM 24734,
Bifidobacterium longum DSM 24736,
Bifidobacterium infantis DSM 24737,
Bifidobacterium breve DSM 24732, and
Streptococcus thermophilus DSM 24731,

4: The method of claim 1, wherein the active probiotic composition comprises:
from between about 20 to 30% by weight of Streptococcus thermophilus,
from between about 20 to 30% by weight of Lactobacillus casei,
from between about 30 to 20% by weight of Bifidobacterium breve, and
from between about 30 to 20% by weight of Bifidobacterium animalis subsp. lactis.

5: The method of claim 1, wherein the inactive composition of probiotics comprises:
Streptococcus thermophilus BT01,
Bifidobacterium breve BB02,
Bifidobacterium longum (recently reclassified as Bifidobacterium animalis subspecies lactis) BL03,
Bifidobacterium infantis (recently reclassified as Bifidobacterium animalis subspecies lactis) B104,
Lactobacillus acidophilus BA05,
Lactobacillus plantarum BP06,
Lactobacillus paracasei BP07, and
Lactobacillus delbrueckii subspecies bulgaricus (recently re classified as Lactobacillus helveticus) BD08.

6: The method of claim 1, wherein the Bacillus amyloliquefaciens is characterized as having a 16S RNA comprising a sequence as set forth in SEQ ID NO:1.

7: An active probiotic composition or an activated inactive probiotic composition comprising a total of between about 2 to 50% by weight of Bacillus amyloliquefaciens and/or Bacillus subtilis.

8: The active probiotic composition or the activated inactive probiotic composition of claim 7, further comprising from between about 98 to 50% by weight of a mixture of the following 8 bacterial strains:
Lactobacillus paracasei DSM 24733,
Lactobacillus plantarum DSM 24730,
Lactobacillus acidophilus DSM 24735,
Lactobacillus delbrueckii subspecies bulgaricus DSM 24734,
Bifidobacterium longum DSM 24736,
Bifidobacterium infantis DSM 24737,
Bifidobacterium breve DSM 24732, and
Streptococcus thermophilus DSM 24731.

9: The active probiotic composition or the activated inactive probiotic composition of claim 7, further comprising:
from between about 20 to 30% by weight of Streptococcus thermophilus,
from between about 10 to 30% by weight of Lactobacillus casei,
from between about 10 to 15% by weight of Bifidobacterium breve,
from between about 10 to 15% by weight of Bifidobacterium animalis subsp. lactis, and
from between about 50 to 10% by weight of Bacillus amyloliquefaciens and/or Bacillus subtilis.

10: The active probiotic composition or the activated inactive probiotic composition of claim 7, comprising from between about 98 to 50% by weight of a mixture of the following bacterial strains:
Streptococcus thermophilus BT01,
Bifidobacterium breve BB02,
Bifidobacterium longum (recently reclassified as Bifidobacterium animalis subspecies lactis) BL03,
Bifidobacterium infantis (recently reclassified as Bifidobacterium animalis subspecies lactis) BI04,
Lactobacillus acidophilus BA05,
Lactobacillus plantarum BP06,
Lactobacillus paracasei BP07, and
Lactobacillus delbrueckii subspecies bulgaricus (recently re classified as Lactobacillus helveticus) BD08.

10: The active probiotic composition or the activated inactive probiotic composition of claim 7, wherein the of Bacillus amyloliquefaciens is characterized as having a 16S RNA comprising a sequence as set forth in SEQ ID NO:1.

10: The active probiotic composition or the activated inactive probiotic composition of claim 7, wherein the active probiotic composition comprises:
from between about 20 to 30% by weight of Streptococcus thermophilus,
from between about 20 to 30% by weight of Lactobacillus casei,
from between about 30 to 20% by weight of Bifidobacterium breve, and
from between about 30 to 20% by weight of Bifidobacterium anima is subsp. lactis.

13: The method of claim 1, wherein the inflammatory bowel disease is selected from the group consisting of: Crohn's disease, ulcerative colitis, indeterminate colitis and microscopic colitis.

14-18. (canceled)

19: The method of claim 13, wherein the inflammatory bowel disease is Crohn's disease.

20: The method of claim 13 wherein the inflammatory bowel disease is ulcerative colitis.

21: The method of claim 13 wherein the inflammatory bowel disease is indeterminate colitis.

22: The method of claim 13, wherein the inflammatory bowel disease is microscopic colitis.
